(19) United States
(12) Patent Application Publication
(10) Pub. No.: US 2026/0029404 A1
(43) Pub. Date: Jan. 29, 2026

(54) Pharmaceutical Composition and Method of Isotope-Selective Modulation

(71) Applicant: ISM BIOSCIENCES LLC, Miami, FL (US)

(72) Inventors: Max Temnik, Miami, FL (US); Oleksandr Balakin, Dnepropetrovsk (UA); Victor Gurin, Richmond, VA (US); Sergey Gurin, Richmond, VA (US)

(73) Assignee: ISM BIOSCIENCES LLC, Miami, FL (US)

(21) Appl. No.: 18/780,726

(22) Filed: Jul. 23, 2024

(51) Int. Cl. G01N 33/58; A61K 45/06; G01N 33/50

(52) U.S. Cl. CPC G01N 33/58; A61K 45/06; G01N 33/5005; G01N 2458/15

(57) ABSTRACT

A pharmaceutical composition for isotope-selective modulation including at least one isotope selected from the group consisting of ¹H, ⁶Li, ¹²C, ¹⁰B, ¹⁴N, ¹⁶O, ¹⁷O, ²⁴ Mg, ²⁶ Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷ Ag, ¹³⁰Ba, ¹³²Ba, ¹³⁴Ba, ¹³⁵Ba, and ¹³⁶Ba, wherein said at least one selected isotope is enriched to exceed its relative isotopic ratio found in a naturally occurring sample of the same chemical element. Methods for its use include investigating the isotopic signatures, intervening with isotopic fractionation in mammals.

### FIELD

This disclosure relates to isotope-selective modulation of biological cells and tissues by application of stable light isotopes of essential chemical elements.

### BACKGROUND

Isotopic signatures, also known as isotopic fingerprints, are the ratios of different isotopes of chemical elements found in a sample material. These ratios can include non-radiogenic ‘stable isotopes’, stable radiogenic isotopes, or unstable radioactive isotopes. Historically, isotopic signatures are used in archeology, forensics, environmental sciences, astrophysics, quantum computing, and food science. However, archeology, forensics, environmental sciences, astrophysics, and food science only use isotopic signatures formed naturally as a result of natural isotopic fractionation, and not purposedly enriched isotopes for investigating and modulating the isotopic signatures.

The use of isotopic signatures in medicine has been limited to application in diagnostics and research. In diagnostic imaging, isotopic signatures is limited to using radioactive isotopes as tracers in medical imaging techniques, for example positron emission tomography (PET) and single-photon emission computed tomography (SPECT), to allow visualization of physiological processes in the body. In these techniques, radioactive isotopes are administered to a patient as radiotracers, where isotopic signature of the radiotracer concentrates in specific organs or tissues based on their biochemical behavior, allowing imaging of those areas.

### SUMMARY

In one aspect, this disclosure provides a pharmaceutical composition for isotope-selective modulation including a prophylactically or therapeutically effective amount of at least one isotope selected from the group consisting of ¹H, ⁶Li, ¹²C, ¹⁰B, ¹⁴N, ¹⁶O, ¹⁷O, ²⁴ Mg, ²⁶ Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba, wherein said at least one selected isotope is enriched to exceed its relative isotopic ratio found in a naturally occurring sample of the same chemical element. In another aspect, this disclosure provides a method of investigating the isotopic signatures including:
(a) Selecting at least one molecule, cell, organelle, and tissue of a biological organ;
(b) Determining isotopic fractionation of at least one isotope selected from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba in at least one region of the selected molecule, cell, organelle, and tissue of a biological organ; and
(c) Assessing isotopic imbalances of at least one isotope selected from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba.

In yet another aspect, this disclosure provides a method of modulating isotopic signatures including:
(a) Selecting at least one molecule, cell, organelle, and/or tissue of a biological organ;
(b) Selecting at least one isotope from the group consisting of ¹H, ⁶Li, ¹²C, ¹⁰B, ¹⁴N, ¹⁶O, ¹⁷O, ²⁴ Mg, ²⁶ Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹ Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba;
(c) Enriching the selected at least one isotope to exceed its relative isotopic ratio found in a naturally occurring sample of the same chemical element, wherein said enrichment is completed on an interrelated or a singular basis; and
(d) Administering a prophylactically or therapeutically effective amount of the enriched selected isotope or combination of isotopes to the selected at least one molecule, cell, organelle, and tissue.

In another aspect, this disclosure provides a method of intervening with isotopic fractionation in mammals including:
(a) Selecting at least one chemical element participating in a biological process or sequence, wherein such chemical element is selected from the group consisting of H, Li, C, B, N, O, Mg, Si, S, K, Cl, Ca, V, Cr, Fe, Ni, Cu, Zn, Mo, Se, Ge, Br, Rb, Ag, and Ba;
(b) Determining interrelation of the selected chemical element to other chemical elements in a biological process or sequence;
(c) Selecting at least one isotope from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵C^{1, 40}Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba;
(d) Enriching the selected at least one isotope to exceed its relative ratio found in naturally occurring sample of the selected chemical element; and
(e) Administering a prophylactically or therapeutically effective amount of the enriched isotope to a mammal orally, rectally, nasally, intramuscularly, intravenously, parenterally, transdermally, sublingually, subcutaneously, intrathecally, buccally, or intravaginally, or a combination thereof.

### BRIEF DESCRIPTION OF DRAWINGS

FIG. 1 is an illustration of isotopic fractionation of stable isotope ⁶⁴Zn in various cells.

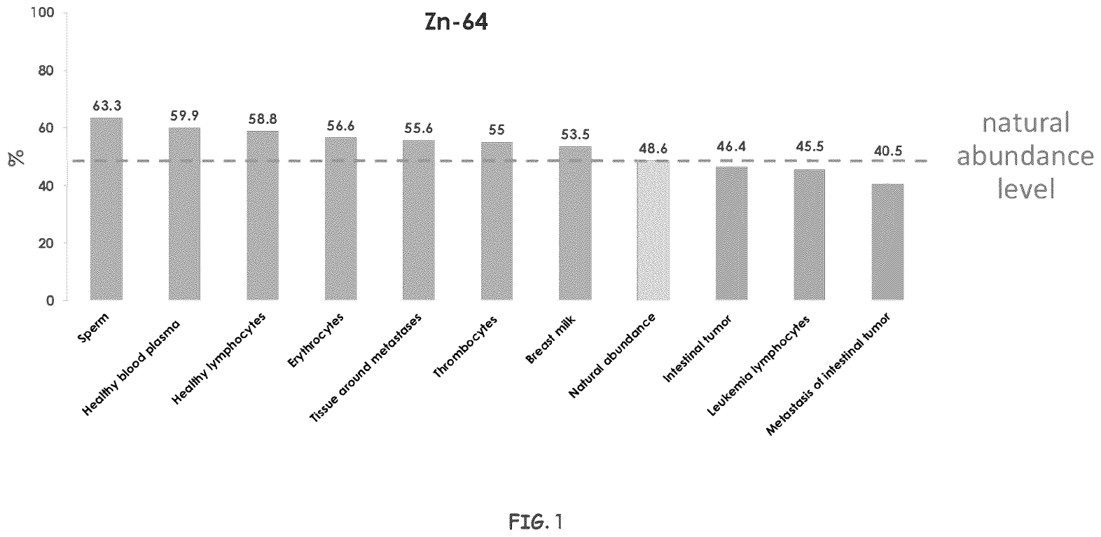

FIG. 2 is an illustration of the method of investigating the isotopic signatures.

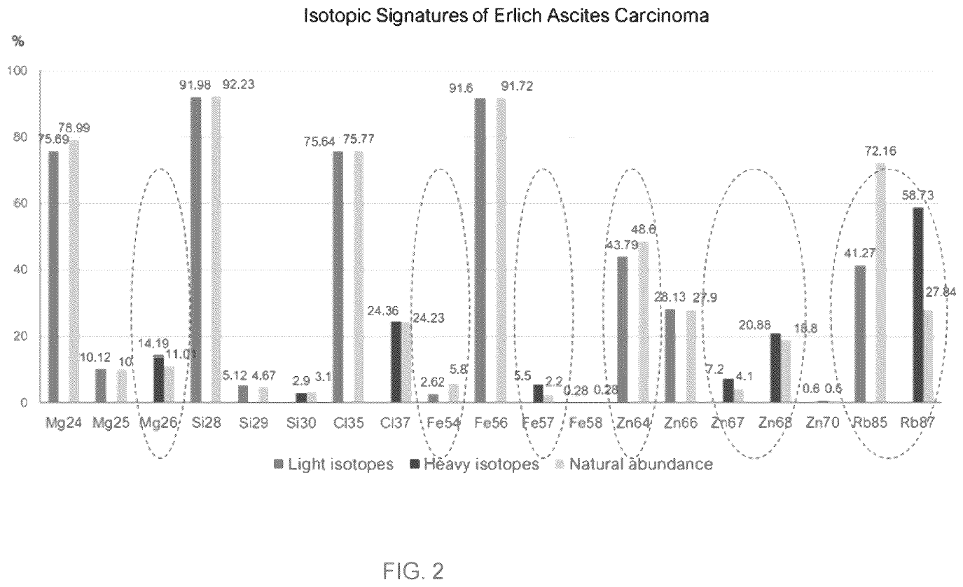

FIG. 3 is an illustration of the method of intervening with and/or slowing down a degenerative progression.

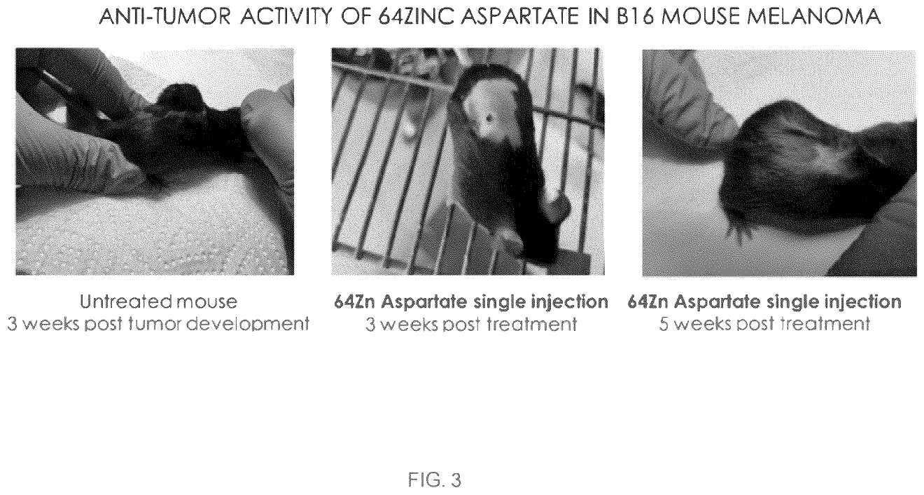

FIG. 4 is an illustration of the composition for isotope-selective modulation.

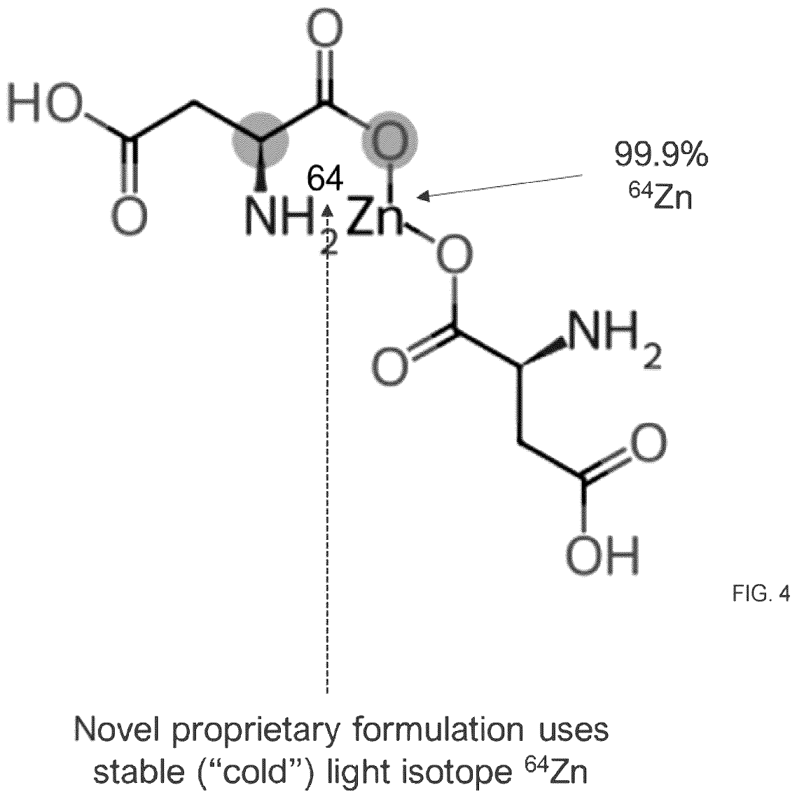

FIG. 5 is an illustration of the method of modulating the isotopic signatures of semisolid agar, A-549, cell colony.

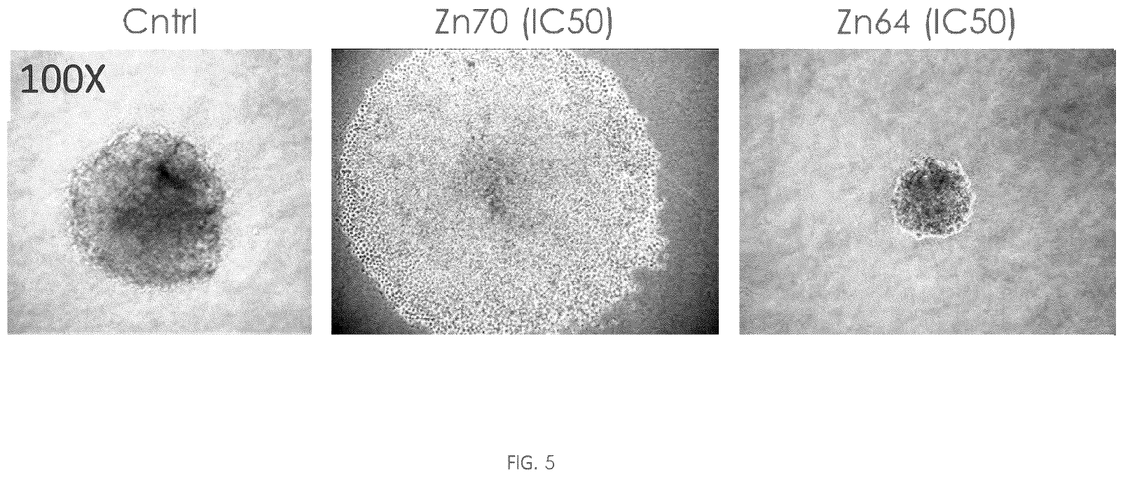

### DETAILED DESCRIPTION

As used herein, the word “a” or “plurality” before a noun represents one or more of the particular noun.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. All ranges disclosed herein are also to be considered to include the end points of the range, unless expressly stated otherwise. For example, a range of “between 5 and 10” or “5 to 10” or “S-10” should be considered to include the end points 5 and 10.

It is further to be understood that the feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. As used herein, the term “isotope” refers to a variant of an atom of a chemical element that differ by the number of neutrons in its nucleus and has a different atomic mass. According to the proton-neutron model developed by D. I. Ivanenko and W. Heisenberg (1932), atoms of all chemical elements consist of three types of elementary particles: positively charged protons, negatively charged electrons, and neutrons that have no charge. The number of protons p in the nucleus determines the atomic number Z of the chemical element in Mendeleev's periodic table The proton and the neutron, which have a common name—nucleons—have almost identical weight. The mass of the neutron (1.00866 amu) is somewhat greater than the proton mass (1.00727 amu). The electron mass is much smaller than that of the nucleons (for example, the proton-to-electron mass ratio is 1836.13). Therefore, the mass of the atom is concentrated in its nucleus. Hence, the mass number of the atom A is connected with the atomic number by a simple relation A=p+n=Z+n, where n is the number of neutrons in the nucleus of an atom. The number of protons in the nucleus of an atom uniquely determines the position of an element in the periodic table of the elements. Furthermore, the number of protons determines the number of electrons present in a neutral atom thus determining the chemical properties of this atom. However, atoms with the same atomic number Z (and hence the number of protons p) may have different neutron numbers n. Thus atoms with different atomic mass numbers may occupy the same position on the periodic table. Chemical elements having the same atomic number but a different atomic mass are known as isotopes.

The term “natural abundance” of an isotope refers to the fraction of the total amount of the corresponding element that the isotope represents, on a mole-fraction basis (that is, not, for example, on a mass basis). For example, if (Zn had an earth natural abundance of 48.63%, that would mean that 48.63% of Zn atoms on earth are the isotope ⁶⁴Zn. When a composition is “enriched” for a certain isotope, the abundance of the isotope in the composition is greater than the isotope's natural abundance For the preceding ⁶⁴Zn example, a composition in which ⁶⁴Zn constitutes more than 48.63% of the total Zn in the composition, on a mole-fraction basis, would be “enriched” for ⁶⁴Zn. Throughout this application, a subscript “e” following a light isotope chemical symbol or element name indicates that the designated element is enriched for that isotope. For example. ⁶⁴Zn refers to the light isotope zinc-64, whereas ⁶⁴Znₑ refers to zine that is enriched for zinc-64. Thus, “⁶⁴Znₑ aspartate,” for example, refers to zinc aspartate in which the zinc is enriched for zinc-64.

The term “relative isotopic ratio” refers to a measure of the proportion of one isotope of a chemical element relative to another isotope of the sample of the same element, typically expressed in comparison to a standard reference. For example, the isotopic ratio of ⁶⁴Zn atoms of naturally occurring zinc is 48.6%.

The term “enriched” refers to the process of increasing the proportional ratio of a specific isotope within a mixture of isotopes. The enrichment process exploits the differences in physical and/or chemical properties between isotopes of the same element to separate and concentrate one isotope from the others.

The term “isotopic imbalance” refers to variations in the isotopic ratios of a particular element within a biological molecule, organelle, cell, or tissues, which deviate from equilibrium values. These imbalances can arise from processes that preferentially select for one isotope over another, leading to changes in the isotopic composition of substances involved in these processes. As used herein, isotopic imbalances are used as indicators of specific biological and/or pathological processes that alter the distribution of isotopes in the molecules, cells, organelles, and/or tissues.

As used herein, the terms “treat,” “treating,” “treatment of” a condition encompass performing an act (such as administering the disclosed composition) in order to cure, eradicate, or diminish the severity of, the condition treated. These terms thus encompass accomplishing any one or more of curing, eradicating, and diminishing the severity of the condition treated.

As used herein, the terms “prevent,” “preventing,” “prevention of” a condition encompass performing an act (such as administering the disclosed composition) in order to prevent the occurrence of the condition and diminish the severity of the condition if it occurs subsequent to the act. These terms thus encompass accomplishing any one or more of wholly preventing the condition from occurring and diminishing the severity of the condition if it occurs subsequent to the act.

“Effective amount,” “prophylactically effective amount,” or “therapeutically effective amount” refers to an amount of an agent or composition that provides a beneficial effect or favorable result to a subject, or alternatively, an amount of an agent or composition that exhibits the desired in vivo or in vitro activity. That result can be reduction, amelioration, palliation, lessening, delaying, and/or alleviation of one or more of the signs, symptoms, or causes of a disease, disorder or condition in a patient/subject, or any other desired alteration of a biological system. An effective amount can be administered in one or more administrations. “Effective amount,” “prophylactically effective amount,” or “therapeutically effective amount” can refer to an amount of an agent or composition that provides a biological, therapeutic, and/or prophylactic result.

The terms “patient” and a “subject” are interchangeable terms and may refer to a human patient/subject, a dog, a cat, a non-human primate, or another kind of mammal.

The term “amino acid” is known in the art. Briefly, it refers to biologically active components that are critical to the structure and function of all living cells. They are organic compounds composed of nitrogen, carbon, hydrogen, and oxygen, along with a variable side chain group. Each amino acid features a central carbon atom (C), known as the alpha (a) carbon, to which an amino group (NH₂), a carboxyl group (COOH), a hydrogen atom (H), and a distinctive side chain (R group) are attached. The chemical nature of the side chain determines the physical and chemical properties of the amino acid, influencing how amino acids interact with each other and with other molecules. Amino acids are the monomers that link together in specific sequences to form proteins and enzymes, which perform a vast array of functions in the body, including catalyzing metabolic reactions, DNA replication, responding to stimuli, and transporting molecules from one location to another. Some amino acids serve as precursors for neurotransmitters. For example, tryptophan is a precursor for serotonin, and tyrosine is a precursor for dopamine and norepinephrine. Some other amino acids like cysteine play a role in the immune system by helping to produce antibodies. Examples of amino acids include but are not limited to alanine, arginine, aspartate, glutamine, histidine, nitrilotriacetic acid (NTA), and leucine.

The term “subclass of amino acids” refers to the categories based on specific characteristics such as structure, polarity, and the chemical properties of their side chains. These classifications help in understanding the roles and functions of amino acids in biological processes, including protein synthesis, enzyme activity, and metabolic pathways. The classification based on structure and polarity includes nonpolar amino acids, polar uncharged amino acids, and charged amino acids. Examples of nonpolar amino acids include glycine, alanine, valine, leucine, isoleucine, methionine, proline, phenylalanine, and tryptophan. These amino acids tend to be located in the interior of proteins, away from the aqueous environment, contributing to the protein's structural stability. Examples of polar, uncharged amino acids include serine, threonine, cysteine, tyrosine. asparagine, and glutamine. These amino acids are often found on the surface of proteins and play roles in enzyme catalysis and substrate binding. Examples of charged amino acids include aspartic acid and glutamic acids, which have negatively charged carboxylate groups, while other examples such as basic amino acids such as lysine, arginine, and histidine have positively charged groups. Charged amino acids are crucial for the function of enzymes, receptors, and ion channels. The classification based on chemical properties of side chains includes aliphatic amino acids, aromatic amino acids, sulfur-containing amino acids, and hydroxyl-containing amino acids. Examples of aliphatic amino acids include glycine, alanine, valine, leucine, and isoleucine. Examples of aromatic amino acids include phenylalanine, tyrosine, and tryptophan that contain an aromatic ring in their side chain, which is important for protein interactions and can absorb ultraviolet light, a feature useful in protein characterization. Examples of sulfur-containing amino acids include methionine and cysteine, which are functional for initiating protein synthesis (methionine) and for maintaining protein structure (cysteine.) Examples of hydroxyl-containing amino acids include serine and threonine, which contain hydroxyl groups in their side chains, are important for phosphorylation, a post-translational modification that regulates protein activity. The term “subclass of amino acids” as used herein further includes special subclasses such as imino acids and conditionally essential amino acids. For example but not limitation, proline is classified as an imino acid due to its cyclic structure, where the amino group is part of the side chain. This unique structure induces kinks in peptide chains and ends alpha helices, hence influencing protein folding. Examples of conditionally essential amino acids include arginine and tyrosine, which are typically synthesized by the body but may need to be ingested under specific conditions such as illness or stress.

The term “variation of amino acid” refers to structural variations in helix subclasses, position-specific amino acid propensities, variations in amino acid sensitivity and detection, and variations in IgG subclasses and allotypes. For instance, certain amino acids like glutamine (Gln) are preferred in all helix subclasses, while others like asparagine (Asn), serine (Ser), and threonine (Thr) are less preferred except in the curved helix subclass, where Asn is favored. Further, the variation in amino acid propensity affects the stability and curvature of helices, impacting protein folding and interaction capabilities. For example, in the regular α-helix subclass, small polar amino acids such as Ser, Thr, Asn, and Aspartic acid (Asp) are preferred at the N1 position, indicating their influence on helix initiation and stability. Conversely, aliphatic amino acids like alanine (Ala), isoleucine (Ile), and leucine (Leu) are favored at the C4 position, contributing to the hydrophobic core and structural integrity of the proteins.

The term “enzyme” is known in the art. Briefly, it refers to a biological catalyst that accelerates chemical reactions within living organisms without being consumed or permanently altered by the reaction. Enzymes function by lowering the activation energy required for a reaction to proceed, thereby increasing the reaction rate and allowing cellular processes to occur efficiently and rapidly under mild conditions. Each enzyme is specific to a particular reaction or type of reaction, a property derived from its unique three-dimensional structure. Examples of enzymes include but are not limited to digestive enzymes (pepsin, amylase, lactase, etc.), metabolic enzymes (creatine kinase, etc.), miscellaneous enzymes (catalase, Lysozyme, etc.)

The term “peptide” is known in the art. Briefly, it refers to a short chain of amino acids linked by peptide bonds, which are formed through a dehydration synthesis reaction between the carboxyl group of one amino acid and the amino group of another. Peptides play various roles in the body. acting as hormones, neurotransmitters, growth factors, and antibiotics, among other functions. They are crucial for many biological processes, including cell signaling, immune responses, and metabolism. Examples of biologically active peptides include insulin, which regulates glucose levels in the blood; glucagon, which has the opposite effect of insulin; and oxytocin, which is involved in childbirth and emotional bonding. The term “peptide” as used in this present invention includes polypeptides.

The term “anion transporting polypeptide” refers to a membrane transport protein that facilitates the cellular uptake of a wide range of organic anions, including various endogenous substances like bile acids, steroid hormones, and thyroid hormones, as well as exogenous substances such as drugs and toxins. Organix anion transporting polypeptides are encoded by the SLCO gene family and are characterized by their ability to transport large and amphipathic molecules across cell membranes in an ATP-independent manner, often functioning as electroneutral exchangers or facilitated transporters. They are expressed in various tissues, including the liver, kidney, intestine, and brain, impacting the pharmacokinetics and pharmacodynamics of many drugs, which makes them significant in the context of biological interactions. One example of an anion transporting polypeptide is OATP1B1. This transporter is primarily located in the liver on the basolateral membrane of hepatocytes and plays a significant role in the hepatic uptake of a wide range of substrates from the blood into the liver cells, contributing to the metabolism and biliary excretion of various endogenous compounds such as bile acids, thyroid hormones, and steroid hormone conjugates, as well as exogenous substances including drugs like statins.

The term “protein” is known in the art. Briefly, it refers to a distinct class of biological molecules due to their ability to coagulate or flocculate under treatments with heat or acid. Proteins are essential for the structure, function, and regulation of the body's tissues and organs. As used herein, term “proteins” further refers to structural proteins, transport proteins, hormonal proteins, defense proteins, storage proteins, contractile proteins, receptor proteins, globular proteins, and fibrous proteins. Examples of structural proteins include collagen and elastin found in connective tissues and keratin found in hair and nails. Examples of transport proteins are hemoglobin, which transports oxygen through the blood, and membrane transport proteins like the GLUT4 transporter. Example of hormonal proteins is insulin, which regulates glucose metabolism by controlling the blood-sugar concentration. Example of defense proteins is immunoglobulin, which attacks and neutralizes pathogens such as bacteria and viruses. Casein in milk and ovalbumin in egg whites are examples of storage proteins that provide nutrients. Examples of contractile proteins include actin and myosin, which are involved in muscle contraction and movement. Keratin is an example of fibrous proteins.

The term “oligonucleotide” is known in the art. Briefly, it refers to a short nucleic acid chain, usually consisting of up to approximately 20 nucleotides. These sequences can be composed of DNA, RNA, or their analogs and are typically synthesized by polymerizing individual nucleotide precursors. Oligonucleotides are crucial in molecular biology and medicine for their ability to bind specifically to complementary nucleotide sequences, influencing gene expression and regulation. Examples include antisense oligonucleotides (ASOs), small interfering RNAs (siRNAs), microRNA (miRNA), molecular probes, aptamers, triplex-forming oligonucleotides (TFOs), locked nucleic acids (LNAs), morpholinos, spiegelmers, and PCR primers.

The term “antibody” is known in the art. Briefly, it refers to specialized Y-shaped immunoglobulins produced by the immune system to identify and neutralize foreign substances such as bacteria, viruses, fungi, and toxins. Antibodies play a crucial role in the body's defense mechanism by recognizing and binding to specific antigens, which are molecules on the surface of pathogens or foreign particles. Examples include immunoglobulins G (IgG), IgM, IgA, IgE, and IgD.

The term “metal-ion binder” refers to a chemical component that works by binding to metal ions, forming stable, water-soluble complexes with metal ions through coordinate or covalent bonds. Examples include ethylenediaminetetraacetic acid (EDTA), dimercaprol (British Anti-Lewisite or BAL), deferoxamine, penicillamine, and succimer (DMSA).

The term “salt” refers to organic and inorganic salts are two broad categories of active compounds that play significant roles in various biological processes. Organic salts are characterized by the presence of carbon-hydrogen (C—H) bonds within their molecular structure. These salts typically result from the reaction of organic acids with bases. The cation (positively charged ion) in these salts often includes organic groups, which can significantly influence the properties and applications of the salts. Inorganic salts do not contain carbon-hydrogen bonds. They are typically formed by the reaction of inorganic acids with bases. The ions in inorganic salts can include metals or other elements from across the periodic table, leading to a vast array of compounds with diverse properties. The fundamental differences between organic and inorganic salts lie in their chemical structure and resultant physical properties. Organic salts, with their organic cations or anions, often participate in organic reactions and have specific uses in organic synthesis and pharmaceutical formulations. In contrast, inorganic salts, with their diverse range of cations and anions, are pivotal in processes that require high thermal stability and solubility in water. Examples of organic salts include but are not limited to sodium acetate (CH₃COONa), potassium citrate (K₃C₆H₅O₇), magnesium stearate (C₃₆H₇₀MgO₄), and benzalkonium chloride (C₂₂H₄₀ClN). Examples of inorganic salts include but are not limited to sodium chloride (NaCl), potassium sulfate (K₂SO₄), magnesium sulfate (MgSO₄), and zinc oxide (ZnO).

The term “metalloenzyme inhibitor” refers to an organic compound that targets metalloenzymes, which are enzymes that require metal ions to function properly. These inhibitors are significant in therapeutic applications, particularly in treating diseases where metalloenzymes play a crucial role. Metalloenzymes incorporate metal ions in their active sites, which are essential for their catalytic activity. These enzymes are involved in a wide range of biological processes, such as metabolism, DNA synthesis, and the regulation of gene expression. Common metallomic elements found in these enzymes include zinc, iron, copper, manganese, and others. Recent research has focused on improving the efficacy and selectivity of metalloenzyme inhibitors through advanced screening techniques and better understanding of metalloenzyme biology. Examples of metalloenzyme inhibitors include but are not limited to carbonic anhydrase inhibitors, matrix metalloproteinases (MMPs) inhibitors, angiotensin-converting enzyme (ACE) inhibitors, histone deacetylase (HDAC) inhibitors, and zinc-containing metalloenzymes inhibitors.

The term “microspheres” refer to spherical particles ranging in size from 1 to 1000 micrometers and used for drug delivery. These particles can be made from various materials, including natural and synthetic polymers, and are designed to encapsulate drugs, providing controlled and sustained release. The use of microspheres in pharmaceuticals offers several advantages, including improved drug stability, targeted delivery, and enhanced patient compliance. Examples include biodegradable microspheres made from the materials such as polylactic acid (PLA), polyglycolic acid (PGA), and their copolymers (PLGA); non-biodegradable microspheres, magnetic microspheres, and floating microspheres.

The term “polymeric micelle” refers to nanoscale colloidal carriers formed by the self-assembly of amphiphilic block copolymers in aqueous solutions. Polymeric micelles have emerged as a significant tool in the field of drug delivery, particularly for the treatment of cancer, due to their unique core-shell structure that enables them to solubilize hydrophobic drugs, enhance drug stability, and facilitate targeted delivery. Examples include NK105 usually incapsulating chemotherapy drugs (i.e. Paclitaxel), NK012 formed from PEG-b-poly (1-glutamic acid) and containing Irinotecan; and NK911 for carrying Doxorubicin-loaded polymeric micelles using PEG-b-poly (α,β-aspartic acid).

The term “liposomes” refers to spherical vesicles composed of one or more phospholipid bilayers, which can encapsulate both hydrophilic and hydrophobic drugs. Examples include small unilamellar vesicles (SUVs), large unilamellar vesicles (LUVs), and multilamellar vesicles (MLVs).

The term “nanosomes” refers to nanocarriers used in precision nanomedicine to deliver therapeutic drugs to specific cells or tissues. Nanosomes have a unique structure consisting of a liposomal bilayer around a hydrophilic core, which can encapsulate either therapeutic drugs or functional biomolecules. This structure allows them to pass through biological barriers and target specific cells or tissues, thereby reducing the side effects associated with traditional drug delivery systems. Examples include nanosome minoxidil, nanosomes carrying doxorubicin, and nanosome-based topical treatments.

The term “adeno-associated virus” (AAV) refers to small, non-enveloped viruses that belong to the Parvoviridae family and the Dependoparvovirus genus. They have gained significant attention in the field of gene therapy due to their unique properties, including low immunogenicity, the ability to infect both dividing and non-dividing cells, and the capacity for long-term gene expression without integrating into the host genome.

The term “metal conjugate” refers to conjugation of metal-based compounds with drugs, polymers, or other carriers to improve drug delivery and therapeutic outcomes. Examples include metal nanoparticles (MNPs), metal-organic frameworks (MOFs), metallopolymers, and metal-based antibody drug conjugates (ADCs).

The term “molecule” is known in the art. Briefly, it refers to an electrically neutral group of two or more atoms held together by chemical bonds. Molecules form the smallest identifiable unit of a chemical compound that retains the composition and chemical properties of that compound. Examples include carbohydrates, lipids, and ribonucleic acid.

The term “cell” is known in the art. Briefly, it refers to the fundamental building blocks of the mammalian body, performing a wide range of functions necessary for life. Each cell type is specialized to carry out specific tasks, contributing to the overall function and maintenance of the body. Examples include stem cells, erythrocytes, leukocytes, myocytes, neurons, and adipocytes.

The term “organelle” is known in the art. Briefly, it refers to specialized structures within cells that perform distinct functions necessary for cellular operation and overall organismal health. These organelles are often compared to organs in the body, each with specific roles that contribute to the cell's survival and functionality. Examples include mitochondria, endoplasmic reticulum, lysosomes, ribosomes, and cytoskeleton.

The term “tissue” is known in the art. Briefly, it refers to groups of cells that work together to perform specific functions. They form intermediate level of organization between cells and organs, playing a crucial role in the structure and function of the mammal body. Examples include epithelial, connective, muscle, and nervous tissues.

The term “organ” is known in the art. Briefly, it refers to complex structure within a living organism that is composed of multiple tissue types working together to perform specific functions essential for the organism's survival and well-being. Organs are integral components of organ systems, which are groups of organs that collaborate to carry out major physiological processes. Examples include the heart, kidney, liver, and the pancreas.

The term “metallome” refers to all metal and metalloid-containing molecules in a biological system and their distribution, isotopes (atoms), and chemical forms (species) of metal ions and inorganic elements bound to proteins, enzymes, nucleic acids, and other biomolecules.

The term “non-metallome” refers to all non-metal and non-metalloid-containing molecules in a biological system and their distribution, isotopes (atoms), and chemical forms (species) of metal ions and inorganic elements bound to proteins, enzymes, nucleic acids, and other biomolecules. For the purpose of clarity, the disclosed composition and methods include isotopes of metallome and non-metallome elements.

Isotopic signatures, also known as isotopic fingerprints, are the ratios of different isotopes of chemical elements found in a natural sample material. These ratios can include non-radiogenic ‘stable isotopes’, stable radiogenic isotopes, or unstable radioactive isotopes.

Stable isotopes are also a known matter. Although radioactive isotopes have been used in medicine for diagnostics and chemotherapies, the use of stable isotopes has been limited to the applications in diagnostics, clinical pharmacology, and nutritional studies, all of which have used stable isotopes in their naturally abundant ratios. In diagnostics, stable isotopes have been used as tracers for dynamically assessing in vivo metabolism. In research, stable isotopes have been used in evaluating bioavailability and the release profile of drug products and drug delivery systems. In the assessment of drug pharmacology, stable isotopes have been used for the determination of a drug's pharmacokinetic profile, mechanism of action, and potential toxicity or adverse effects. In precision medicine, stable isotopes have recently started to be used monitoring drug treatment effects and conducting clinical toxicology studies. In nutrition, stable isotopes have been used to assess body composition, energy expenditure, protein turnover, food safety and metabolic profiles. However, all of these prior art techniques and technologies have used stable isotopes in their naturally occurring isotopic ratios.

Isotope enrichment is also a known process that refers to the process of increasing the proportion of a specific isotope in a mixture of isotopes. The enriched stable isotopes of carbon (C), nitrogen (N), and oxygen (O) in both medicine and pharmaceutical applications have been disclosed. These enriched isotopes were used for various purposes, including drug development and diagnostics. For instance, isotopes ¹³C and ¹⁵N have been used in clinical medicine and biological studies. They are particularly valuable in the development of diagnostic tests, such as the ¹³C urea breath test for detecting Helicobacter pylori infections. Also, the chemical compounds labeled with highly enriched ¹³C are used in breath tests for diagnosing liver and intestine diseases.

Isotopic signatures are the ratios of different isotopes found in a sample of a chemical element. These ratios can include non-radiogenic stable isotopes, stable radiogenic isotopes, or unstable radioactive isotopes. The measurement of these ratios is typically conducted using isotope-ratio mass spectrometry, which compares the isotopic composition of the sample against a reference material.

Changes in isotopic signatures is a dynamic, ever-present process occurring in biological organisms far before the degeneration of tissues begins. Maintaining the isotopic homeostasis (both metallome and non-metallome) within biological organisms is essential to healthy cellular and organ functioning. Maintaining such isotopic homeostasis is further essential not only for treatment of diseases, but more so for preventing physiological dysfunctions leading to the diseases. For example, superoxide dismutase 1 (SOD1) is a crucial enzyme encoded by the SOD1 gene located on chromosome 21922.11. This enzyme plays a vital role in protecting cells from oxidative damage by catalyzing the conversion of superoxide radicals into molecular oxygen and hydrogen peroxide. SOD1 is a homodimeric enzyme, meaning it consists of two identical subunits, each containing a copper and a zinc ion essential for its catalytic activity. Both copper and zinc are metallome elements, which atoms (isotopes) defer by the number of neutrons in their nucleuses.

Stable isotopes of zinc are ⁶⁴Zn with natural isotopic ratio of 48.6%, ⁶⁶Zn with natural isotopic ratio of 27.9%, ⁶⁷Zn with natural isotopic ratio of 4.10%, ⁶⁸Zn with natural isotopic ratio of 18.75%, and ⁷⁰Zn with natural isotopic ratio of 0.62%. Stable isotopes of Cu are ⁶³Cu with natural isotopic ratio of 69.17% and ⁶⁵Cu with natural isotopic ratio of 30.83%. When natural zinc and copper in this example are administered into mammal organisms through dietary and environmental intake, their isotopes participate in biochemical reactions. Because biochemical reactions involve kinetic isotope effects, where the reaction parameters differ for molecules containing lighter versus heavier isotopes, and other factors, the natural abundance of isotopes after intake changes, leading to isotopic fractionation within the organisms. The same rationale applies to other metallome and non-metallome elements.

Once translated, the incipient monomeric polypeptide of SOD1 binds to one zinc atom providing structural integrity before the direct interaction with copper chaperone for superoxide dismutase (CCS) which ensures the binding of copper atom required for catalytic activity in biochemical reactions. The establishment of a properly functional SOD1 enzyme then occurs through intramolecular disulfide bond formation and combination with another monometric protein to create the functional homodimer. Enzymes can cause isotopic fractionation by preferentially catalyzing reactions involving lighter isotopes. This is due to the lower activation energy required for breaking bonds with lighter isotopes.

The establishment of properly functional SOD1 enzyme may be induced by supplying the lightest zinc atoms (isotopes) for the reaction, in which case the translated SOD1 will preferentially bind to the zinc atoms featuring the kinetic factors favoring the autocatalytic reaction, hence binding selectively to the lightest zinc atoms. Because of kinetic isotope effect, binding selectively to the lightest zinc atoms will provide for the faster/easier sequential reactions within the organism. At the same time, because physical parameters of zinc are the same across all its isotopes, the structural integrity of SOD1 enzyme will remain unchanged. The binding of the incipient monomeric polypeptide of SOD1 to light stable zinc isotopes will also occur easier and faster due to the kinetic isotope effect. The isotopic signatures of the resulting functional homodimer may be modulated by selectively supplying light atoms of zinc. Supplying zinc and copper enriched of light atoms may induce healthy stabilization of the native structure of SOD1, hence promoting homo-dimerization and leading to ensuring structural integrity of properly folded SOD1, resulting in lesser unfolding. Hence, the isotope-selective modulation of SOD1 folding and binding processes may prevent the mutations of SOD1, leading to lesser oxidative damage and induced healthy cellular function.

This disclosure relates to using enriched stable isotopes for modulating the isotopic signatures of biological molecules, cells, organelles, and tissues, which can be used for preventive and therapeutic treatments. Although a therapeutic effect can be derived from a therapeutic application, the modulation of isotopic signatures in general is also intended as a preventive measure.

The disclosed pharmaceutical composition includes an isotope-selective modulator in form of the metallomic and/or non-metallomic element in which the isotopic distribution of at least one light isotope atom is altered from that of naturally abundant distribution ratio to that of exceeding its natural ratio (that isotope atom is said to be enriched; that is existing at an abundance greater than its natural amount). The disclosed pharmaceutical composition does not require presence of any other substance except the at least one isotopes as disclosed herein. The disclosed pharmaceutical composition does not require a combination of the isotope-selective component with at least one amino acid, chelator, metal-ion binder, salt, and/or metalloenzyme inhibitor, though embodiments herein can further comprise them. The chelators that can be used with the isotope-selective modulator are not limited to those containing a ligand bonded to a central metal atom at at least two points.

The isotope-selective modulation can be performed by supplying a stable light isotope to off-set the accumulation of stable heavy isotopes in mammal organisms. Furthermore, the isotope-selective modulation of isotopic signatures is interrelated and does not have to be absolute, meaning that modulating a selected isotope-specific signature by supplying one stable isotope may lead to the changes in other isotopic signatures. For example, an excretion of ⁶⁵Cu from the mutant liver tissue of Wilson's Disease patient may be achieved by modulating the isotopic signatures of such mutation by administering selectively ⁶⁴Zn isotope not bound to but combined with an amino acid. Alternatively, such isotope-selective modulation may be performed by administering said ⁶⁴Zn isotope in colloidal form, depending on the disease phase, patient specifics, isotope-selective bioavailability profile, and other case-specific factors. In one aspect, this disclosure provides a pharmaceutical composition for isotope-selective modulation including a prophylactically or therapeutically effective amount of at least one isotope selected from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba, wherein said at least one selected isotope is enriched to exceed its relative isotopic ratio found in a naturally occurring sample of the same chemical element. In certain embodiments, the pharmaceutical composition further includes a prophylactically or therapeutically effective amount of at least one amino acid, a subclass or a variation of amino acid, an enzyme, a peptide, an anion transporting polypeptide, a protein, a protein nanocage, an oligonucleotide, an antibody, or a combination thereof. In certain embodiments, the pharmaceutical composition further includes at least one metal-ion binder, a salt, and/or a metalloenzyme inhibitor, a microsphere, a polymeric micelle, a liposome, a nanosome, an adeno-associated virus, a metal conjugate, or a combination thereof.

This disclosure provides a method of modulating the isotopic signatures of biological cells and tissues to prevent or correct a disruption of essential physiological processes and/or cure abnormalities and diseases. The method begins with investigating the isotopic signatures of targeted cells and/or tissues. The method continues with modulating the investigated isotopic signatures by administering at least one isotope-selective modulator selected from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba, or a combination thereof. The bioavailability, cellular uptake, and absorption speed of the isotope-selective modulator can be improved by administering such component in combination with at least one amino acid, chelator, metal-ion binder, salt, metalloenzyme inhibitor, and/or another organic compound or pharmaceutical agent.

This disclosure also provides a method of intervening with, slowing down, or possibly halting a isotopic fractionation process. The method begins with investigating the subject process for a dependency on metallomic element deficiency. The method further includes investigating the subject process for a dependency on isotopic sensitivity. The method concludes with administering an isotope-selective compound containing at least one metallomic element selected from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵C^{1, 40}Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba, or a combination thereof, where said element features stable light isotope atoms enriched to exceed their respective natural abundance ratios.

In another aspect, this disclosure provides a method of investigating the isotopic signatures including:

(a) Selecting at least one molecule, cell, organelle, and/or tissue of a biological organ; and
(b) Determining isotopic fractionation of at least one isotope selected from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba in at least one region of the selected molecule, cell, organelle, and tissue of a biological organ.
(c) Assessing isotopic imbalances of at least one isotope selected from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba.

In another aspect, this disclosure provides a method of modulating isotopic signatures including:
(a) Selecting at least one molecule, cell, organelle, and/or tissue of a biological organ;
(b) Selecting at least one isotope from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba;
(c) Enriching the selected at least one isotope to exceed its relative isotopic ratio (weighted average percentage of atoms with a specific atomic mass found in a naturally occurring sample of said element) found in a naturally occurring sample of the same chemical element, wherein said enrichment is completed on an interrelated or a singular basis; and
(d) Administering a prophylactically or therapeutically effective amount of the enriched selected isotope or combination of isotopes to the selected at least one molecule, cell, organelle, and tissue.

In some embodiments of the method, administering said enriched isotope is conducted prior to, concurrent with, or after administering a known drug, organic compound, or medicinal substance to said organelles, molecules, cells, and tissues, wherein the enriched isotope can be in its elemental form or as an ingredient of the drug, compound, or medicinal substance. In certain embodiments, the assessment of isotopic imbalance is conducted in relation to other isotopes selected from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba. In certain embodiments, the assessment of isotopic imbalance is considered in regard to the biological homeostasis of a subject organism.

In another aspect, this disclosure provides a method of intervening with isotopic fractionation including:
(a) Selecting at least one chemical element participating in a biological process or sequence, wherein such chemical element is selected from the group consisting of H, Li, C, B, N, O, Mg, Si, S, K, Cl, Ca, V, Cr, Fe, Ni, Cu, Zn, Mo, Se, Ge, Br, Rb, Ag, and Ba;
(b) Determining interrelation of the selected chemical element to other chemical elements in a biological process or sequence;
(c) Selecting at least one isotope from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba:

(d) Enriching the selected at least one isotope to exceed its relative ratio found in naturally occurring sample of the selected chemical element;
(e) Administering a prophylactically or therapeutically effective amount of the enriched isotope to a mammal orally, rectally, nasally, intramuscularly, intravenously, parenterally, transdermally, sublingually, subcutaneously, intrathecally, buccally, and/or intravaginally, or via a combination thereof.

The “at least one isotope” can be one or more, up to all the isotopes, disclosed in the disclosed composition or method.

Using one isotope to modulate an isotopic signature can lead to homeostatic changes of the element's heavier isotopes, which must be considered on a case-by-case basis using the precision medicine principles and mechanisms of action, especially when the isotope-selective modulation is used in combination therapies.

The disclosed isotope-selective component may be administered orally, rectally, nasally, intramuscularly, intravenously, parenterally, transdermally, sublingually, subcutaneously, intrathecally, buccally, and/or intravaginally.

Metallomic elements, including both macro and trace minerals, are integral to cellular function. These elements are involved in nearly every aspect of physiological function, from metabolic processes and structural integrity to antioxidant defense and immune function. They serve as cofactors for enzymes, structural components of proteins and membranes, and signaling molecules that regulate cellular activities.

The supply of metallomic and other essential chemical elements to biological cells, tissues, and organs is crucial for sustaining life and promoting health. Ensuring adequate uptake is vital for preventing deficiencies and associated health issues. This highlights the interconnectedness of nutrition, metabolism, and overall health, underscoring the importance of a balanced intake of these essential elements.

Metallomic elements, which include both essential and trace metals, are crucial for many biological processes, including enzymatic reactions, structural functions, and cellular signaling. Many enzymes require metal ions as cofactors to function correctly. For example, zinc is involved in the metabolism of proteins, carbohydrates, and lipids and is crucial for DNA synthesis and cell division and is a component of over 300 enzymes involved in digestion, metabolism, and DNA and tissue repair.

Iron is central to the function of cytochromes in the electron transport chain and is crucial for energy production in cells. Metals like calcium and magnesium play significant structural roles in the body. Calcium is vital for bone health and neuromuscular function, while magnesium is involved in over 300 biochemical reactions in the body, including those that stabilize DNA structures. Certain metals, such as iron and copper, are essential for neurological function. Iron is crucial for the synthesis of neurotransmitters, and copper plays a role in brain development and maintenance certain metallomic elements are involved in the body's defense against oxidative stress. Selenium, for example, is a core component of glutathione peroxidase, an enzyme that protects cells from damage by reactive oxygen species. Copper and manganese are also part of superoxide dismutase, which helps in dismantling harmful superoxide radicals.

The depletion of metallomic elements from biological cells, tissues, and organs of mammals can be attributed to a variety of causes, ranging from dietary intake to environmental factors and genetic disorders. The depletion of metallomic elements from biological cells and tissues is a natural process that can disrupt critical biological functions, leading to a range of health issues from metabolic disorders to immune deficiency and neurological problems. Aging and specific health conditions can also impact the absorption and regulation of metallomic elements. For example, the efficiency of intestinal uptake of some trace metals, particularly zinc, declines in the elderly. Additionally, diseases affecting the kidneys, liver, or gastrointestinal tract can impair the body's ability to maintain adequate levels of essential metals. Furthermore, an intake of several medications and medical treatments can also lead to the depletion of metallomic elements. For example, long-term use of some diuretics and other medications can increase the excretion of metals like magnesium and potassium, leading to deficiencies.

With the exception of mono-isotopic elements, the metallomic elements consist of two or more isotopes. Isotopes are variants of a particular chemical element that differ in the number of neutrons, and hence in nucleon number, but share the same number of protons. This isotopic variation is fundamental to the field of metallomics, especially when considering the application of stable isotope compositions as tracers for studying the metabolism of metal elements in various biological samples, including tissues from plants, animals, and humans.

The study of metallomics integrates techniques and perspectives from other “-omics” sciences, such as genomics and proteomics, and applies them to bio-metal research. This interdisciplinary approach has led to the development of isotope metallomics, which focuses on the distribution and effects of stable metal isotopes within biological systems.

Isotopic fractionation refers to the small changes in the isotope composition of elements during chemical reactions or physical processes. In the human body, this can occur during metabolic activities such as absorption, transport, and incorporation of metals into biological molecules. The fractionation of isotopes in the human body primarily results from enzymatic activities or transport processes that prefer lighter isotopes due to their lower mass and higher reaction rates. For example, lighter isotopes might be processed faster or bond more readily in certain biochemical reactions.

Isotopic fractionation is common for many elements in the human body. For example, it has been demonstrated by a number of studies that the isotopic fractionation in cells, tissues, and organs can serve as a diagnostic marker. In particular, the study of the ratios of Cu and Zn isotopes in blood showed their promising interrelationships with age, sex and pathologies. For example, an estimate of the ratio of Cu isotopes in blood serum is a new approach to the diagnosis and prognosis of the development of cirrhosis (M. Costas-Rodriguez, Y. Anoshkina, S. Lauwens, H. Van Vlierberghe, J. Isotopic analysis of Cu in blood serum by multi-collector ICP-mass spectrometry: a new approach for the diagnosis and prognosis of liver cirrhosis, Metallomics 2015, 7. 491-498), and the isotopic composition of Zn in breast tissues enables diagnosis of cancer (F. Larner, L. N. Woodley, S. Shousha, A. Moyes, E. Humphreys-Williams, S. Strekopytov, A. N. Halliday, M. Rchkamper, R. C. Coombes, Zinc isotopic compositions of breast cancer tissue, Metallomics 2015, 7. 107-112).

Isotopic fractionation has high physiological relevance to several diseases. For instance, variations in the isotopic composition of copper and zinc have been associated with cancer and metabolic disorders. Studies have shown that the isotopic composition of Cu and Zn in serum can differ significantly between healthy individuals and those with diseases such as cancer. The isotopic compositions of ⁶³Cu, ⁶⁴Zn/⁶⁶Zn, ²⁴ Mg/²⁶ Mg and ⁵⁴Fe/⁵⁶Fe in the brain changes with age, which might be linked to neurodegenerative diseases. Hence, the replenishment of the depleted isotopes in biological cells, tissues, and organs represents a cutting-edge approach that leverages the subtle differences in the isotopic composition of elements in the body to enhance weakening biological functions. The application of isotopic fractionation in drug development offers unique opportunities in the pharmaceutical industry for the development of novel precision medicines featuring enhanced efficacy and safety.

Malignant tissues are enriched with heavy isotopes of chemical elements, and the healthy tissues surrounding the malignant tissues are enriched with light isotopes. The body may be attempting to fight the malignancies by altering the isotopic ratios of chemical elements found in the healthy tissues surrounding the malignancies in favor of the light isotopes. The inventors also assumed that the malignant cells and tissues may feed out of accumulating heavy isotopes of the chemical elements. Consequently, the inventors have researched the role of the isotopic composition in atoms of metallomic elements plays in physical, chemical, and biological processes and their effect on cell death, tissue degeneration, and other abnormalities.

Administering heavy stable isotopes to malignant oncological cells leads to active proliferation of such cells. Administering light stable isotopes to the malignant cells leads to their shrinkage. Altering the isotopic signatures of metallomic elements through administration of stable light isotopes renders a therapeutic effect on sick cells while not harming healthy cells.

The disclosed composition may be presented in various dosage forms depending on the object of application; in particular, it may be formulated as a solution for injections.

The disclosed composition can be administered to a subject in need thereof by any suitable mode of administration, any suitable frequency, and at any suitable, effective dosage.

The disclosed composition may be in any suitable form and may be formulated for any suitable means of delivery.

The composition may be administered systemically. Suitable routes of administration include, for example, oral or parenteral administration, such as intravenous, intraperitoneal, intragastric as well as via drinking water. However, depending on a dosage form, the disclosed composition may be administered by other routes.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

What is claimed is:

1. A pharmaceutical composition for isotope-selective modulation including a therapeutically or prophylactically effective amount of at least one isotope selected from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba, wherein said at least one selected isotope is enriched to exceed its relative isotopic ratio found in a naturally occurring sample of the same chemical element.

2. The pharmaceutical composition of claim 1 further including a therapeutically or prophylactically effective amount of at least one amino acid, a subclass or a variation of amino acid, an enzyme, a peptide, an anion transporting polypeptide, a protein, a protein nanocage, an oligonucleotide, an antibody, or a combination thereof.

3. The pharmaceutical composition of claim 1 further including at least one metal-ion binder, a salt, and/or a metalloenzyme inhibitor, a microsphere, a polymeric micelle, a liposome, a nanosome, an adeno-associated virus, a metal conjugate, or a combination thereof.

4. A method of investigating isotopic signatures including:
(a) Selecting at least one molecule, cell, organelle, and/or tissue of a biological organ; and
(b) Determining isotopic fractionation of at least one isotope selected from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba in at least one region of the selected molecule, cell, organelle, and tissue of a biological organ.
(c) Assessing isotopic imbalances of at least one isotope selected from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba.

5. The Method of claim 4, wherein the assessment of isotopic imbalances is conducted in relation to other isotopes selected from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba.

6. The Method of claim 4, wherein the assessment of isotopic imbalances is considered in regard to the biological homeostasis of a subject molecule, cell, organelle, and/or tissue and physiological processes in which said selected at least one isotope is involved.

7. A method of modulating isotopic signatures including:
(a) Selecting at least one molecule, cell, organelle, and/or tissue of a biological organ;
(b) Selecting at least one isotope from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba;
(c) Enriching the selected at least one isotope to exceed its relative isotopic ratio found in a naturally occurring sample of the same chemical element, wherein said enrichment is completed on an interrelated or a singular basis; and
(d) Administering the enriched selected isotope or combination of isotopes to the selected at least one molecule, cell, organelle, and/or tissue to isotopically modulate physiological processes in which said at least one molecule, cell, organelle, and/or tissue is involved.

8. The method of claim 7, wherein administering said enriched isotope is conducted prior to. concurrent with, or after administering therapeutically or prophylactically effective amount of a known drug, organic compound, or medicinal substance to said organelles, molecules, cells, and tissues, wherein the enriched isotope is in its elemental form or as an ingredient of the drug, compound, or medicinal substance.

9. A method of intervening with isotopic fractionation including:
(a) Selecting at least one chemical element participating in a biological process or sequence, wherein such chemical element is selected from the group consisting of H, Li, C, B, N, O, Mg, Si, S, K, Cl, Ca, V, Cr, Fe, Ni, Cu, Zn, Mo, Se, Ge, Br, Rb, Ag, and Ba;
(b) Determining interrelation of the selected chemical element to other chemical elements in a biological process or sequence;
(c) Selecting at least one isotope from the group consisting of ¹H, ⁶Li, ¹²C, ¹⁰B, ¹⁴N, ¹⁶O, ¹⁷O, ²⁴Mg, ²⁶Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁷⁹Br, ⁸⁵Rb, ¹⁰⁷Ag, ¹³⁰Ba, ¹³²Ba, ¹³⁴Ba, ¹³⁵Ba, and ¹³⁶Ba;
(d) Enriching the selected at least one isotope to exceed its relative ratio found in naturally occurring sample of the selected chemical element; and
(e) Administering a prophylactically or therapeutically effective amount of the enriched isotope to a mammal orally, rectally, nasally, intramuscularly, intravenously, parenterally, transdermally, sublingually, subcutaneously, intrathecally, buccally, and/or intravaginally, or via a combination thereof.
